(19) United States
(12) Patent Application Publication
(10) Pub. No.: US 2020/0050718 A1
(43) Pub. Date: Feb. 13, 2020

(54) SIMULATION APPARATUS, SIMULATION METHOD, AND STORAGE MEDIUM

(71) Applicant: NEC Corporation, Minato-ku, Tokyo (JP)

(72) Inventors: Yuya YAMAKAWA, Tokyo (JP); Katsuhiro OCHIAI, Tokyo (JP)

(73) Assignee: NEC Corporation, Minato-ku, Tokyo (JP)

(21) Appl. No.: 16/343,032

(22) PCT Filed: Oct. 31, 2016
(86) PCT No.: PCT/JP2016/004763

§ 371 Date: Apr. 18, 2019

(51) Int. Cl. G06F 17/50; G01N 33/24

(52) U.S. Cl. CPC G06F 17/5009; G01N 2033/245; G01N 33/246

(57) ABSTRACT

A simulation apparatus, a simulation method, and a storage medium that can implement a water flow simulation with higher accuracy compared to the conventional water flow simulator are provided. The simulation apparatus has an estimated value acquisition unit that calculates an estimated value of soil water content in a target region, a determination unit that determines a parameter in consideration of an error between the estimated value of the soil water content in the target region and an actual measurement value of the soil water content in the target region, and a simulator unit that performs a simulation of a water flow in the target region by using the parameter.

### TECHNICAL FIELD

The present invention relates to a simulation apparatus, a simulation method, and a storage medium that predict a water flow, which is a flow of water in a target region.

### BACKGROUND ART

Technologies for monitoring a condition of a river, an embankment, or the like and predicting a flood of the river and a collapse of the embankment have been developed. Among these technologies, there is a technology that predicts a runoff amount of a target river by performing a water flow simulation using a rainfall prediction and then predicts a flood or an embankment breakdown based on the result thereof.

Such a technology requires a water flow simulator to perform a water flow simulation. The water flow simulator consists of two models: a distributed runoff model and a river channel kinematic wave model. The distributed runoff model is a model used to calculate a water flow in a region other than the river in the target region. The river channel kinematic wave model is a model used to calculate a water flow of the river area in the target region. There are a plurality of parameters in these models, and the appropriate setting of these parameters enables a more realistic water flow simulation. Currently, there is a technology that compares an actual measurement value of the river runoff amount with a river runoff amount calculated by the water flow simulation to determine an appropriate parameter. However, there are not many parameter determination methods that use the actual measurement values other than the river runoff amount.

### CITATION LIST

### Patent Literature

PTL 1: Japanese Patent Application Laid-Open No. 2009-008651

### SUMMARY OF INVENTION

### Technical Problem

In a water flow simulation using a water flow simulator, a target region is divided into meshes, a rainfall prediction falling on each mesh is used as input, and thereby a runoff amount at any timing in any location is calculated. Since the parameters in the distributed runoff model and the river channel kinematic wave model described above are parameters related to geographical features such as a stratum thickness, a roughness coefficient, or a hydraulic conductivity coefficient, a more realistic simulation can be performed by appropriate setting of these parameters.

As a determination method for the parameters described above, there is currently a method that evaluates an error to an actual measurement value of a river flow amount. In this determination method, specifically, several parameter candidates for the parameter described above are first generated from field survey, literature survey, empirical rules, or the like and then used to perform a water flow simulation. Next, the actual measurement value measured by the river runoff amount meter and the simulation result at the installation site of the river runoff amount meter are compared as an error. As a result of the comparison, a parameter in which the simulation result is the closest to the actual measurement value is adopted as a true parameter.

The problem in the simulation described above is that a simulation error can be evaluated only at an installation site of a river runoff amount meter. That is, the simulation can only improve the accuracy of the river channel kinematic wave model used for performing the water flow simulation of the river area. Therefore, since a calculation error of the distributed runoff model that calculates a runoff amount at a region where water flows into the river cannot be directly evaluated, the input error to the river channel kinematic wave model cannot be minimized.

Accordingly, when actual measurement data such as soil water that can be measured in a region other than a river can be used to improve the accuracy of the distributed runoff model, the problem described above can be solved. However, since the result calculated in the distributed runoff model is a runoff amount in each mesh and a result corresponding to a value of the soil water meter is not output, the direct evaluation cannot be performed. It is therefore difficult to determine more appropriate parameters by reflecting the actual measurement value of the soil water meter to the conventional simulation.

The example object of the present invention is to provide a simulation apparatus, a simulation method, and a storage medium that can realize a water flow simulation with higher accuracy than to the conventional water flow simulator.

### Solution to Problem

According to an example aspect of the present invention, provided is a simulation method that calculates an estimated value of soil water content in a target region, determines a parameter in consideration of an error between the estimated value of the soil water content in the target region and an actual measurement value of the soil water content in the target region, and performs a simulation of a water flow in the target region by using the parameter.

According to another example aspect of the present invention, provided is a simulation apparatus including an estimated value acquisition unit that calculates an estimated value of soil water content in a target region, a determination unit that determines a parameter in consideration of an error between the estimated value of the soil water content in the target region and an actual measurement value of the soil water content in the target region, and a simulator unit that performs a simulation of a water flow in the target region by using the parameter.

According to yet another example aspect of the present invention, provided is a storage medium that stores a program that causes a computer to perform steps of calculating an estimated value of soil water content in a target region, determining a parameter in consideration of an error between the estimated value of the soil water content in the target region and an actual measurement value of the soil water content in the target region, and performing a simulation of a water flow in the target region by using the parameter.

### Advantageous Effects of Invention

According to the present invention, a water flow simulation with high accuracy compared to the conventional water flow simulator can be realized.

### BRIEF DESCRIPTION OF DRAWINGS

FIG. 1 is a block diagram illustrating a functional configuration of a simulation apparatus according to an example embodiment of the present invention.

FIG. 2 is a schematic diagram illustrating river channel sections.

FIG. 3 is a schematic diagram illustrating water flow directions in meshes.

FIG. 4 is a schematic diagram illustrating a soil water content.

FIG. 5 is a flowchart illustrating an operation flow of the simulation apparatus according to an example embodiment of the present invention.

FIG. 6 is a block diagram illustrating a functional configuration of a simulation apparatus according to another example embodiment of the present invention.

### DESCRIPTION OF EMBODIMENTS

### One Example Embodiment

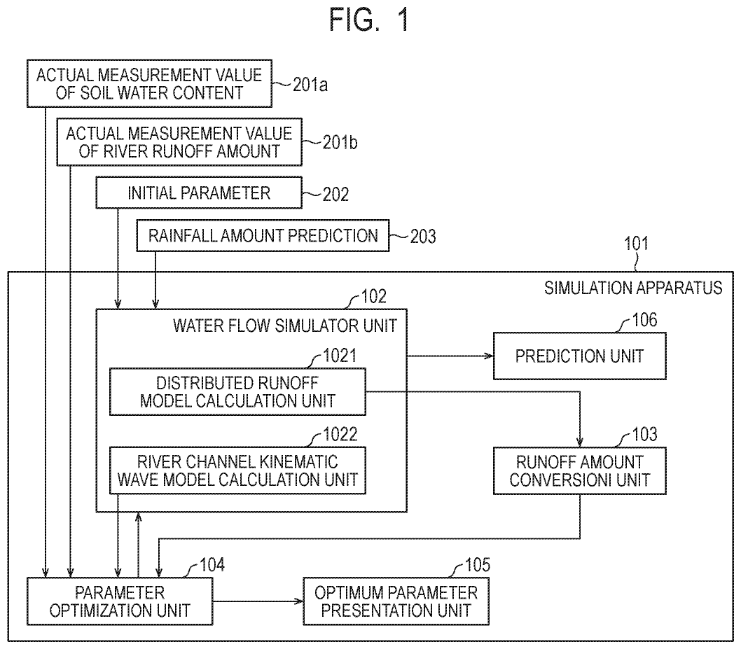

A simulation apparatus and a simulation method according to an example embodiment of the present invention will be described with reference to FIG. 1 to FIG. 5.

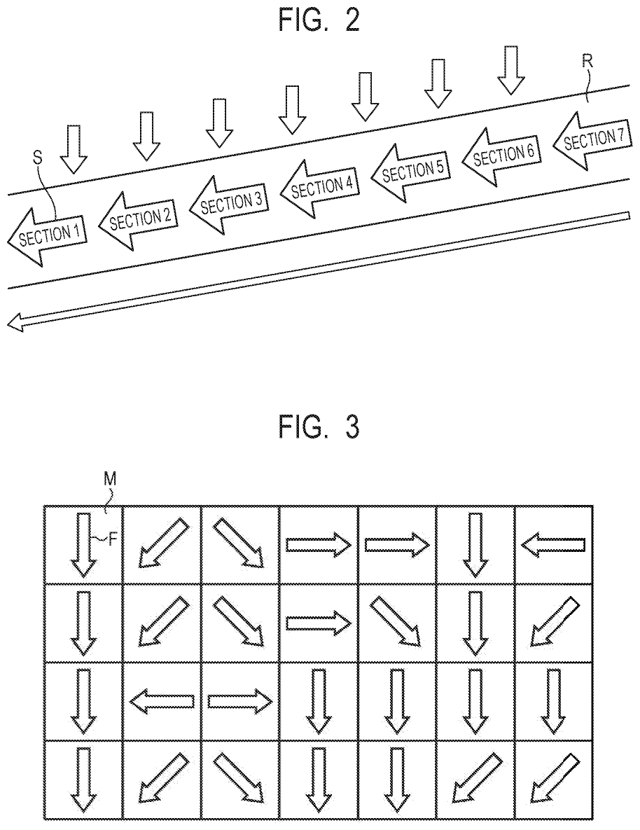

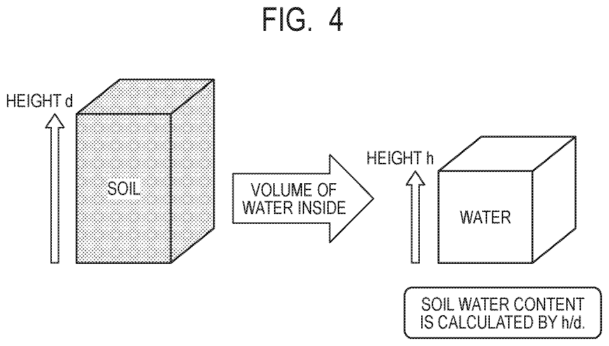

First, a configuration of the simulation apparatus according to the present example embodiment will be described by using FIG. 1 to FIG. 4. FIG. 1 is a block diagram illustrating a functional configuration of the simulation apparatus according to the present example embodiment. FIG. 2 is a schematic diagram illustrating the river channel sections. FIG. 3 is a schematic diagram illustrating water flow directions in the meshes. FIG. 4 is a schematic diagram illustrating a soil water content.

The simulation apparatus according to the present example embodiment is a simulation apparatus that predicts a water flow, which is a flow of water in a target region, by using a water flow simulator and includes a parameter automatic determination function that automatically determines a parameter in the water flow simulation performed by the water flow simulator. The parameter automatic determination function uses an actual measurement value of the soil water content, an actual measurement value of the river runoff amount, an initial parameter, and a rainfall amount prediction as input to automatically determine the optimum parameter by which the water flow simulator can represent the actual situation with the highest accuracy. Here, the initial parameter is a parameter that is set in the distributed runoff model and the river channel kinematic wave model of the water flow simulator, and an operator of the water flow simulation sets the appropriate one. The parameter relates to the geographical features such as a stratum thickness, a roughness coefficient, or a hydraulic conductivity coefficient, or the like, for example. Here, the person who sets the parameter is not required to have special knowledge of the parameter setting method.

As illustrated in FIG. 1, the simulation apparatus 101 according to the present example embodiment includes a water flow simulator unit 102, a runoff amount conversion unit 103, a parameter optimization unit 104, an optimum parameter presentation unit 105, and a prediction unit 106. The water flow simulator unit 102 includes a distributed runoff model calculation unit 1021 and a river channel kinematic wave model calculation unit 1022. Note that each one-directional arrow between respective blocks in FIG. 1 indicates a direction of a signal or a data flow in a simplified manner and is not intended to exclude the bidirectionality of the signal or data flow.

The water flow simulator unit 102 functions as a simulator unit that simulates a water flow in the target region using a parameter. The water flow simulator unit 102 generates the time-series runoff amount data in the target region of the water flow simulation by using a preset initial parameter 202 and a rainfall amount prediction 203 as input. The water flow simulation in the target region by using the initial parameter 202 is the initial simulation. Here, the river channel kinematic wave model calculation unit 1022 calculates a water flow in the river area of the target region by using the river channel kinematic wave model. On the other hand, the distributed runoff model calculation unit 1021 calculates a water flow in the area other than the river in the target region by using the distributed runoff model.

The river channel kinematic wave model in which the river channel kinematic wave model calculation unit 1022 used for water flow calculation of the river area is an algorithm that calculates an input runoff amount to the downstream river section at time t as follows. That is, as illustrated in FIG. 2, a river R in a target region is divided into a plurality of river sections S in the river channel kinematic wave model. FIG. 2 illustrates a case where the river R is divided into a section 1 to a section 7 as the river sections. The water flow calculation is then performed on a divided river section basis by being input with a rainfall falling into the river at the time t, an inflow amount flowing into the river from the region other than the river at the time t, and the initial parameter. In such a way, the input runoff amount to a downstream river section at the time t is calculated.

On the other hand, the distributed runoff model in which the distributed runoff model calculation unit 1021 used for water flow calculation of the area other than the river is an algorithm that calculates a runoff amount to a lower mesh at the time t as follows. That is, for example, the target region is divided into meshes of 50 m square or 250 m square in the distributed runoff model. As illustrated in FIG. 3, with respect to a runoff amount F calculated in each mesh M, it is determined in advance that whole the water flows into adjacent meshes having the greatest difference in height. Based on such a condition, a runoff amount to the lower mesh at the time t is calculated with an amount prediction of rainfall falling into each mesh at the time t, an initial parameter, and an inflow amount from an upper mesh at the time t being used as input.

In general, when the water flow simulation by using the water flow simulator is performed, a parameter setting in the distributed runoff model and the river channel kinematic wave model constructing the water flow simulator may be a factor that significantly affects the result. Since the accuracy of the water flow simulation changes depending on the parameter setting, when the improvement of the accuracy of the water flow simulation is considered, how to set an appropriate parameter setting is the key.

As described above, when a parameter can be determined by reflecting an actual measurement value of the river runoff amount and an actual measurement value of the soil water content in the region other than the river, a more accurate water flow simulator in which the accuracy is higher than that of a conventional water flow simulator can be constructed. Here, as described above, a value corresponding to the soil water content is not calculated in an output results of the water flow simulator. It is therefore difficult to determine a parameter by reflecting an actual measurement value of the soil water content measured by the soil water meter to perform an error evaluation.

On the other hand, in the present example embodiment, the runoff amount conversion unit 103 described below converts an estimated value of the runoff amount at the installation site of the soil water meter output from the distributed runoff model calculation unit 1021 of the water flow simulator unit 102 into an estimate value of the soil water content at the installation site of the soil water meter. Note that a calculation method of the estimated value of the soil water content will be described in detail below. This enables evaluation of an error between the estimated value of the soil water content and an actual measurement value of the soil water content measured by the soil water meter in the present example embodiment. By formulating the evaluation of the error between the estimated value of the soil water content and the actual measurement value of the soil water content into an optimization problem for determining an optimum parameter, solving the optimization problem will correspond to determination of the optimum parameter. The optimization problem will be solved in the parameter optimization unit 104 described below. In the present example embodiment, since the parameter of the water flow simulation is determined by reflecting not only the actual measurement value of the river runoff amount but also the actual measurement value of the soil water content, a water flow simulation with high accuracy compared to the conventional water flow simulator can be realized.

The runoff amount conversion unit 103 is input with a runoff amount at the time t in the mesh of the installation site of the soil water meter calculated by the distributed runoff model calculation unit 1021 and converts the runoff amount thereof into an estimated value of a soil water content at the time t. Note that the soil water meter is installed in an area other than the river in the target region. As described above, the runoff amount conversion unit 103 functions as an estimated value acquisition unit that acquires an estimated value of the soil water content of the target region based on the calculation result of the water flow in an area other than the river in the target region calculated by the distributed runoff model calculation unit 1021.

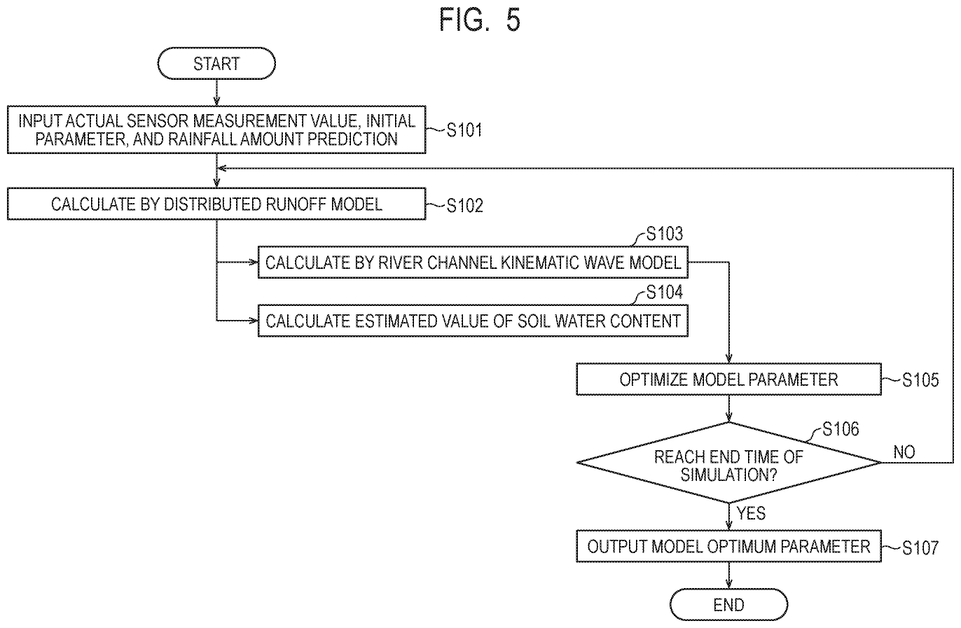

Here, the soil water content is calculated as a volume of water contained per unit volume of the soil as described in FIG. 5. That is, the runoff amount is converted into the estimated value of the soil water content by calculating the volume of the water contained in a mesh at the installation site of the soil water meter and dividing the calculated volume of the water by the volume of the mesh (soil volume) at the installation site of the soil water meter. Here, since both of the mesh sizes are, for example, 50 m square or 250 m square, the soil water content is equal to the value h/d obtained by dividing the water level h contained in the mesh by the soil stratum thickness d of the mesh. The soil stratum thickness of the mesh is regarded as a parameter that does not change during the simulation. Therefore, determining the water level h contained in the mesh corresponds to determining the soil water content.

Accordingly, a method for determining the water level contained in a mesh will be described below. The distributed runoff model is based on the following Equation (1).

$$\begin{matrix}[ Math . 1 ] & \\ \{ \begin{matrix}\frac{\partial h}{\partial t} + \frac{\partial q}{\partial x} = r \\ h = {Φ}_{θ} ( q )\end{matrix} & ( 1 )\end{matrix}$$

In Equation (1), h is a water level in the mesh, q is a runoff amount of the water flowing out of the mesh, r is a rainfall amount, and ϕ_{θ} is a function that has θ as a parameter and q as a variable. Note that there are several patterns in the form of ϕ_{θ} depending on the modeling method. In the distributed runoff model, the runoff amount q is calculated by solving both the equations. Therefore, by using the second equation in Equation (1), the water level h in the corresponding mesh can be obtained by calculating ϕ_{θ}(q) with the runoff amount q. Note that, while being treated as a fixed value using the initial parameter when the runoff amount q described above is calculated, the parameter θ is treated as a variable when the water level h of the mesh. The reason why the parameter θ is treated as a variable when the water level h is calculated is to obtain the optimum parameter by performing an optimization in the parameter optimization unit 104 described below.

Note that the estimated value of the soil water content can also be calculated as follows. That is, since the soil water content is the volume of the water content per unit volume of soil, a total water content flowing into the installation site of the soil water meter is first calculated from the rainfall amount falling into the installation site of the soil water meter and the water content flowing in from the upstream area. Next, the runoff amount at the installation site of the soil water meter obtained by the water flow simulation is subtracted from the total water content described above. The amount obtained by subtraction in such a way is the water content at the installation site of the soil water meter. On the other hand, the volume of the mesh at the installation site of the soil water meter is calculated by multiplying the square of the mesh size (50 m square or 250 m square, for example) by the vertical height (soil stratum thickness). The estimated value of the soil water content at the installation site of the soil water meter can be calculated by dividing the water content at the installation site of the soil water meter by the volume of the mesh at the installation site of the soil water meter.

The parameter optimization unit 104 functions as a determination unit that automatically determines an optimum parameter by correcting the parameter of the distributed runoff model and the river channel kinematic wave model. As described below, when determining the optimum parameter, the parameter optimization unit 104 takes into consideration of an error between the estimated value of the soil water content and the actual measurement value of the soil water content measured by the soil water meter.

First, the parameter optimization unit 104 receives the estimated value of the soil water content at the time t from the runoff amount conversion unit 103 and an actual measurement value 201a of the soil water content measured by the soil water meter at the time t to be input. The soil water meter is installed in an area other than the river in the target region. In such a way, the parameter optimization unit 104 also functions as an actual measurement value acquisition unit that acquires an actual measurement value of the soil water content in the target region. The parameter optimization unit 104 then determines an optimum parameter of the distributed runoff model at the time t by solving the appropriate optimization problem by using the received estimated value of the soil water content and the actual measurement value of the soil water content. As described above, taking into consideration of an error between the estimated value of the soil water content and the actual measurement value of the soil water content measured by the soil water meter, the parameter optimization unit 104 determines the optimum parameter of the distributed runoff model.

Further, the parameter optimization unit 104 receives the estimated value of the runoff amount at the site where the river runoff amount meter exists at the time t calculated by the river channel kinematic wave model calculation unit 1022 and an actual measurement value 201b of the river runoff amount at the time t to be input measured by the river runoff amount meter. Then, the parameter optimization unit 104 also determines an optimum parameter of the river channel kinetic wave model at the time t by solving the appropriate optimization problem by using the received estimated value of the runoff amount and the actual measurement value of the river runoff amount.

In the following, first, a method for determining an optimum parameter of the distributed runoff model in the parameter optimization unit 104 will be described. An actual measurement value of the soil water content provided as input is denoted as m. Here, with the parameter θ that minimizes the error between the estimated value ϕ_{θ}(q)/d of the soil water content described above calculated from a result of the water flow simulation and the actual measurement value m of the soil water content, it can be said that the simulation well represents the actual situation. Therefore, the following formulation will be performed.

[Math. 2]

minimize |f(θ)−m|² (2)

subject to θ ∈ Θ

In Equation (2), although being equal to ϕ_{θ}(q)/d, f(6) is denoted as f(θ) in order to express ϕ_{θ}(q)/d as a function of the parameter θ, which is a variable. Further, Θ is a set for providing a constraint condition of the parameter θ, which is a variable. Since some parameters have constraints such as not being able to take negative values, not being over a certain value, or the like, such a situation is addressed by an appropriate usage of Θ. In the optimization problem in Equation (2), the error between the estimated value and the actual measurement value of the soil water content at the time t is a target function, which is to be minimized under the appropriate constraint condition Θ. Solving this corresponds to determining a parameter such that the estimated value approaches the actual measurement value. Then, the optimum parameter of the distributed runoff model is determined by solving the optimization problem formulated as with Equation (2). Next, as for a method for determining an optimum parameter of the river channel kinematic wave model in the parameter optimization unit 104, a formulation as with Equation (2) may be performed based on the same idea as the determination method of the optimum parameter of the distributed runoff model described above. Specifically, in determination of the optimum parameter in the river channel kinematic wave model, the function f(θ) in Equation (2) is replaced with a runoff amount obtained when the parameter θ at the time t calculated by the simulation is a variable. In addition, the value m in Equation (2) is replaced with an actual measurement value of the river runoff amount. Further, the set Θ in Equation (2) is appropriately set in consideration of the range that may be taken by the parameter of the river channel kinematic wave model.

Next, a method for solving the optimization problem of the Equation (2) will be described. A general mathematical optimization method can be used for the solution. As the solution, for example, the main dual interior point method, the sequential quadratic program method, the extended Lagrange method, or the like can be used. By using such an optimization method, the optimization problem for optimizing a parameter of the distributed runoff amount and the optimization problem for optimizing a parameter of the river channel kinematic wave model are solved. The set of two parameters obtained in such ways is determined as the optimum parameters at the time t output from the parameter optimization unit 104. When the time t at which the optimum parameter is obtained is not the end time in the simulation period, the water flow simulator unit 102 performs a water flow simulation by using the optimum parameters obtained at this time t for the next simulation time. On the other hand, when the time t at which the optimum parameter is obtained is the end time in the simulation period, the parameter optimization unit 104 delivers the optimum parameter to the optimum parameter presentation unit 105.

Here, as a specific example, determination of an optimum parameter of the distributed runoff model in the case where calculation is performed by using the following Equation (3) in the distributed runoff model calculation unit 1021 of the water flow simulator unit 102 will be described. Equation (3) is an equation that uses a specific function as ϕ_{θ} in Equation (1). Note that, as described above, the optimum parameter of the river channel kinematic wave model can also be determined in the same idea.

$$\begin{matrix}[ Math . 3 ] & \\ \frac{\partial h}{\partial t} + \frac{\partial q}{\partial x} = r \\ h = {Kq}^{α} & ( 3 )\end{matrix}$$

In Equation (3), h is a water level in the mesh, q is a runoff amount of the water flowing out of the mesh, r is a rainfall amount, and K and a are parameters. In the distributed runoff model calculation unit 1021, the runoff amount q is calculated by solving both the equations. The runoff amount conversion unit 103 uses the second equation in Equation (3) and estimates the soil water content by using the runoff amount q of the simulation result. When the soil stratum thickness is denoted as d here, the estimated value of the soil water content can be expressed as the following Equation (4).

$$\begin{matrix}[ Math . 4 ] & \\ f ( α , K , d ) := \frac{{Kq}^{α}}{d} & ( 4 )\end{matrix}$$

Note that f on the left side of Equation (4) is a function that provides an estimated value of the soil water content when the parameters α, K, d are considered as variables. Therefore, the runoff amount conversion unit 103 estimates a soil water content at the time t in a form including parameters by substituting the runoff amount q of the simulation result at the time t into the right side of Equation (4).

The parameter optimization unit 104 determines an optimized parameter of the distributed runoff model by solving the optimization problem formulated as with the following Equation (5). The optimization problem illustrated in Equation (5) uses an actual measurement value m of the soil water content at the time t and an estimated value of the soil water content at the time t calculated by the runoff amount conversion unit 103.

$$\begin{matrix}[ Math . 5 ] & \\ minimize {\vert \frac{{Kq}^{α}}{d} - m \vert}^{2} \\ subject to ( α , K , d ) ∈ Θ & ( 5 )\end{matrix}$$

Note that, in Equation (5), Θ is a set for providing constraints on parameters α, K, and d considered as variables. Since some parameters have constraint such as not being able to take a negative value, not being over a certain value, or the like, such a condition is addressed by appropriately using Θ. In the optimization problem of Equation (5), an error between the estimated value and the actual measurement value of the soil water content at the time t is denoted as an objective function, which will be minimized under the appropriate constraint condition Θ. Solving this corresponds to determining parameters α, K, and d by which the estimated value approaches the actual measured value. Then, by solving the optimization problem formulated as Equation (5) by using the optimization method described above, the optimum parameter of the distributed runoff model is determined. The optimum parameter calculated in such a way is determined as the output result of the parameter optimization unit 104.

The optimum parameter presentation unit 105 presents the optimum parameters of the distributed runoff model and the optimum parameters of the river channel kinematic wave model calculated in the parameter optimization unit 104 as the optimum parameters in the target region.

The prediction unit 106 predicts an influence by the water flow in the target region based on the calculation result of the water flow in the target region by the water flow simulator unit 102. Specifically, the prediction unit 106 can predict a water level of the river, a soil water content, a slope collapse, a flood, or the like in the target region, for example, as the influence by the water flow.

Note that the simulation apparatus 101 according to the present example embodiment is implemented by a central processing unit (CPU) that executes a process in accordance with a program, for example. Further, the simulation apparatus 101 may be implemented by a computer that includes the CPU and a storage medium for storing a program and operates under the control of the CPU based on the program. Further, the simulation apparatus 101 may be formed of a single apparatus or more than one physically separated apparatuses connected by a wire or wirelessly.

Further, the water flow simulator unit 102, the runoff amount conversion unit 103, and the parameter optimization unit 104 are implemented by the CPU that executes the process in accordance with the program, for example.

Note that a part or whole of the program to be executed by the CPU of the computer can be provided by a computer readable recording medium such as a digital versatile disc-read only memory (DVD-ROM), a compact disc-read only memory (CD-ROM), an universal serial bus (USB) memory, other flash memories, or the like in which the program thereof is stored.

Next, a simulation method by using the simulation apparatus 101 according to the present example embodiment will be further described by using FIG. 5. FIG. 5 is a flowchart illustrating the operation of the simulation apparatus 101 according to the present example embodiment.

The actual measurement value 201a of the soil water content, the actual measurement value 201b of the river runoff amount, the initial parameter 202, and the rainfall amount prediction 203 are input to the simulation apparatus 101 (step S101). The actual measurement value 201a of the soil water content is an actual measurement value of the sensor measured by the soil water meter. The actual measurement value 201b of the river runoff amount is an actual measurement value of the sensor measured by the river runoff amount meter.

The water flow simulator unit 102 acquires model initial value parameters, which are the time-series data of the rainfall amount and the initial values of the model parameters of the water flow simulator, respectively, from the rainfall amount prediction 203 and the initial parameter 202 to be input. The water flow simulator unit 102 performs the initial simulation of the water flow in the target region by using the initial parameter 202 as follows.

The distributed runoff model calculation unit 1021 calculates a runoff amount of each mesh by using the distributed runoff model with the model initial value parameters and the time-series data of the rainfall amount (step S102).

The river channel kinematic wave model calculation unit 1022 calculates a runoff amount of each river channel section by using the river channel kinematic wave model with the model initial value parameters, the time-series data of the rainfall amount, and the calculation result of the distributed runoff model calculation unit 1021 (step S103).

The runoff amount conversion unit 103 calculates an estimated value of the soil water content at the installation site of the soil water meter by using the calculation result of the distributed runoff model calculation unit 1021 (step S104).

The parameter optimization unit 104 determines an optimum parameter of the distributed runoff model by solving the appropriate optimization problem by using the actual measurement value of the soil water content, which is an actual measurement value of the sensor, and the estimated value of the soil water content calculated in the runoff amount conversion unit 103. Further, the parameter optimization unit 104 calculates an optimum parameter of the river channel kinematic wave model by solving the appropriate optimization problem by using the calculation result of the river channel kinematic wave model calculation unit 1022 and the actual measurement value of the river runoff amount, which is the actual measurement value of the sensor. In such a way, the parameter optimization unit 104 optimizes the parameters of the distributed runoff model and the river channel kinematic wave model (step S105).

In the calculation up to step S105, when the time t has not reached the simulation end time (step S106, “No”), the process proceeds to step S102, and the calculation of steps S102 to S105 is repeated again. Here, the parameter optimization unit 104 delivers the parameters of the distributed runoff model out of the optimum parameters obtained in step S105 to the distributed runoff model calculation unit 1021. Further, the parameter optimization unit 104 delivers the parameters of the river channel kinematic wave model out of the optimum parameters obtained in step S105 to the river channel kinematic wave model calculation unit 1022. In such a way, the optimized parameters are reflected in the simulation of the next time in the distributed runoff model calculation unit 1021 and the river channel kinematic wave model calculation unit 1022.

On the other hand, when the time t has reached the simulation end time (step S106, “Yes”), the parameter optimization unit 104 delivers the optimum parameters to the optimum parameter presentation unit 105. The optimum parameter presentation unit 105 outputs the delivered optimum parameters as model optimum parameters (step S107).

The distributed runoff model calculation unit 1021 and the river channel kinematic wave model calculation unit 1022 in the water flow simulator unit 102 can perform the calculation of the water flow by using the model optimum parameters presented by the optimum parameter presentation unit 105, respectively.

Further, based on the calculation result of the water flow of the target region by the water flow simulator unit 102, the prediction unit 106 predicts a water level of the river, a soil water content, a slope collapse, a flood, or the like in the target region, for example, as the influence by the water flow in the target region.

In such a way, in the present example embodiment, the parameters of the water flow simulation by the water flow simulator unit 102 are determined in consideration of not only the actual measurement value of the river runoff amount but also the actual measurement value of the soil water content. Therefore, according to the present example embodiment, a water flow simulation with higher accuracy than the conventional water flow simulator can be realized. Accordingly, by using the water flow simulation result according to the present example embodiment, the accuracy of river water level prediction, slope collapse prediction, soil water content prediction, flood prediction, or the like can be improved compared to prediction by the conventional methods. Note that soil water content prediction can be utilized for agriculture information and communication technology (ICT).

### Another Example Embodiment

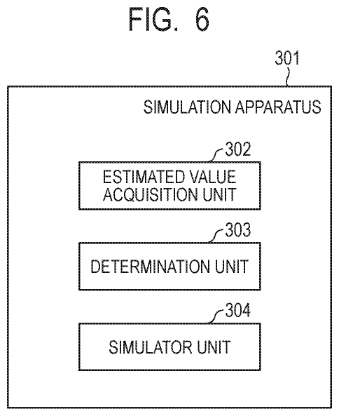

According to another example embodiment, the simulation apparatus illustrated in each example embodiment described above can also be configured as illustrated in FIG. 6. FIG. 6 is a block diagram illustrating a functional configuration of a simulation apparatus according to another example embodiment.

As illustrated in FIG. 6, a simulation apparatus 301 has an estimated value acquisition unit 302 that calculates an estimated value of the soil water content of the target region. Further, the simulation apparatus 301 has a determination unit 303 that determines parameters in consideration of an error between the estimated value of the soil water content of the target region and an actual measurement value of the soil water content of the target region. Further, the simulation apparatus 301 has a simulator unit 304 that performs a simulation of a water flow in the target region by using the parameters.

### Modified Example Embodiments

The present invention is not limited to the example embodiments described above, and various modifications are possible.

In the example embodiments described above, for example, although the case where the water flow in the river area is calculated by the river channel kinematic wave model and the water flow in the area other than the river is calculated by the distributed runoff model in the water flow simulator unit 102 has been described as an example, the present invention is not limited thereto. The water flow in the river area and the water flow in the area other than the river can be calculated by various models, respectively. Further, the water flow simulator unit 102 can also be configured to calculate the water flow of the target region without dividing the river area and the area other than the river, for example.

The whole or part of the example embodiments disclosed above can be described as, but not limited to, the following supplementary notes.

(Supplementary Note 1)

A simulation method comprising:

calculating an estimated value of a soil water content in a target region;

determining a parameter in consideration of an error between the estimated value of the soil water content in the target region and an actual measurement value of the soil water content in the target region; and

performing a simulation of a water flow in the target region by using the parameter.

(Supplementary Note 2)

The simulation method according to supplementary note 1 further comprising:

performing an initial simulation of the water flow in the target region by using an initial parameter; and

calculating the estimated value of the soil water content in the target region by using the initial simulation.

(Supplementary Note 3)

The simulation method according to supplementary note 1 or further comprising determining the parameter so as to reduce the error between the estimated value of the soil water content and the actual measurement value of the soil water content.

(Supplementary Note 4)

The simulation method according to supplementary note 2, wherein the initial simulation comprises

calculating a water flow in an area other than a river in the target region, and

acquiring the estimated value of the soil water content in the target region based on a calculation result of the water flow in the area other than the river.

(Supplementary Note 5)

The simulation method according to supplementary note 4, wherein the actual measurement value of the soil water content is measured by a soil water meter installed in the area other than the river.

(Supplementary Note 6)

The simulation method according to supplementary note 4 or further comprising calculating and acquiring the estimated value of the soil water content by dividing a water level in the area other than the river in the target region by a soil stratum thickness in the area other than the river.

(Supplementary Note 7)

The simulation method according to supplementary note 6 further comprising:

calculating the water flow in the area other than the river by using a distributed runoff model; and

calculating the water level in the area other than the river based on a calculation result by the distributed runoff model.

(Supplementary Note 8)

The simulation method according to any one of supplementary notes 1 to 7 further comprising determining the parameter by solving an optimization problem in which the error between the estimated value of the soil water content and the actual measurement value of the soil water content is a target function.

(Supplementary Note 9)

A simulation apparatus comprising:

an estimated value acquisition unit that calculates an estimated value of a soil water content in a target region;

a determination unit that determines a parameter in consideration of an error between the estimated value of the soil water content in the target region and an actual measurement value of the soil water content in the target region; and

a simulator unit that performs a water flow simulation of a water flow in the target region by using the parameter.

(Supplementary Note 10)

The simulation apparatus according to supplementary note 9,

wherein the simulator unit performs an initial simulation of the water flow in the target region by using an initial parameter, and

wherein the estimated value acquisition unit calculates the estimated value of the soil water content in the target region by using the initial simulation.

(Supplementary Note 11)

The simulation apparatus according to supplementary note 9 or 10, wherein the determination unit determines the parameter so as to reduce the error between the estimated value of the soil water content and the actual measurement value of the soil water content.

(Supplementary Note 12)

The simulation apparatus according to supplementary note 10,

wherein the simulator unit calculates a water flow in an area other than a river in the target region in the initial simulation, and

wherein the estimated value acquisition unit acquires the estimated value of the soil water content in the target region based on a calculation result of the water flow in the area other than the river.

(Supplementary Note 13)

The simulation apparatus according to supplementary note 12, wherein the actual measurement value of the soil water content is measured by a soil water meter installed in the area other than the river.

(Supplementary Note 14)

The simulation apparatus according to supplementary note 12 or 13, wherein the estimated value acquisition unit calculates and acquires the estimated value of the soil water content by dividing a water level in the area other than the river in the target region by a soil stratum thickness in the area other than the river.

(Supplementary Note 15)

The simulation apparatus according to supplementary note 14,

wherein the simulator unit calculates the water flow in the area other than the river by using a distributed runoff model; and

wherein the estimated value acquisition unit calculates the water level in the area other than the river based on a calculation result by the distributed runoff model.

(Supplementary Note 16)

The simulation apparatus according to any one of supplementary notes 9 to 15, wherein the determination unit determines the parameter by solving an optimization problem in which the error between the estimated value of the soil water content and the actual measurement value of the soil water content is a target function.

(Supplementary Note 17)

A storage medium that stores a program that causes a computer to perform:

calculating an estimated value of a soil water content in a target region;

determining a parameter in consideration of an error between the estimated value of the soil water content in the target region and an actual measurement value of the soil water content in the target region; and

performing a simulation of a water flow in the target region by using the parameter.

### REFERENCE SIGNS LIST

101 simulation apparatus
102 water flow simulator unit
103 runoff amount conversion unit
104 parameter optimization unit
105 optimum parameter presentation unit
106 prediction unit
1021 distributed runoff model calculation unit
1022 river channel kinematic wave model calculation unit

What is claimed is:

1. A simulation method comprising:
calculating an estimated value of a soil water content in a target region;
determining a parameter in consideration of an error between the estimated value of the soil water content in the target region and an actual measurement value of the soil water content in the target region; and
performing a simulation of a water flow in the target region by using the parameter.

2. The simulation method according to claim 1 further comprising:
performing an initial simulation of the water flow in the target region by using an initial parameter; and
calculating the estimated value of the soil water content in the target region by using the initial simulation.

3. The simulation method according to claim 1 further comprising determining the parameter so as to reduce the error between the estimated value of the soil water content and the actual measurement value of the soil water content.

4. The simulation method according to claim 2, wherein the initial simulation comprises
calculating a water flow in an area other than a river in the target region, and
acquiring the estimated value of the soil water content in the target region based on a calculation result of the water flow in the area other than the river.

5. The simulation method according to claim 4, wherein the actual measurement value of the soil water content is measured by a soil water meter installed in the area other than the river.

6. The simulation method according to claim 4 further comprising calculating and acquiring the estimated value of the soil water content by dividing a water level in the area other than the river in the target region by a soil stratum thickness in the area other than the river.

7. The simulation method according to claim 6 further comprising:
calculating the water flow in the area other than the river by using a distributed runoff model; and
calculating the water level in the area other than the river based on a calculation result by the distributed runoff model.

8. The simulation method according to claim 1 further comprising determining the parameter by solving an optimization problem in which the error between the estimated value of the soil water content and the actual measurement value of the soil water content is a target function.

9. A simulation apparatus comprising:
an estimated value acquisition unit that calculates an estimated value of a soil water content in a target region;
a determination unit that determines a parameter in consideration of an error between the estimated value of the soil water content in the target region and an actual measurement value of the soil water content in the target region; and
a simulator unit that performs a water flow simulation of a water flow in the target region by using the parameter.

10. The simulation apparatus according to claim 9,
wherein the simulator unit performs an initial simulation of the water flow in the target region by using an initial parameter, and
wherein the estimated value acquisition unit calculates the estimated value of the soil water content in the target region by using the initial simulation.

11. The simulation apparatus according to claim 9, wherein the determination unit determines the parameter so as to reduce the error between the estimated value of the soil water content and the actual measurement value of the soil water content.

12. The simulation apparatus according to claim 10,
wherein the simulator unit calculates a water flow in an area other than a river in the target region in the initial simulation, and
wherein the estimated value acquisition unit acquires the estimated value of the soil water content in the target region based on a calculation result of the water flow in the area other than the river.

13. The simulation apparatus according to claim 12, wherein the actual measurement value of the soil water content is measured by a soil water meter installed in the area other than the river.

14. The simulation apparatus according to claim 12, wherein the estimated value acquisition unit calculates and acquires the estimated value of the soil water content by dividing a water level in the area other than the river in the target region by a soil stratum thickness in the area other than the river.

15. The simulation apparatus according to claim 14,
wherein the simulator unit calculates the water flow in the area other than the river by using a distributed runoff model; and
wherein the estimated value acquisition unit calculates the water level in the area other than the river based on a calculation result by the distributed runoff model.

16. The simulation apparatus according to claim 9, wherein the determination unit determines the parameter by solving an optimization problem in which the error between the estimated value of the soil water content and the actual measurement value of the soil water content is a target function.

17. A non-transitory storage medium that stores a program that causes a computer to perform:
calculating an estimated value of a soil water content in a target region;
determining a parameter in consideration of an error between the estimated value of the soil water content in the target region and an actual measurement value of the soil water content in the target region; and
performing a simulation of a water flow in the target region by using the parameter.
